(19) United States
(12) Patent Application Publication
(10) Pub. No.: US 2016/0103112 A1
(43) Pub. Date: Apr. 14, 2016

(54) PLANT SEED COMPUTER MORPHOMETRY TO DETERMINE DARWINIAN VARIATION FITNESS

(71) Applicant: Irwin Rovner, Raleigh, NC (US)

(72) Inventor: Irwin Rovner, Raleigh, NC (US)

(21) Appl. No.: 14/513,383

(22) Filed: Oct. 14, 2014

(51) Int. Cl. G01N 33/00; H04N 7/18; G01N 21/25; G01B 11/02; G01B 11/30

(52) U.S. Cl. CPC G01N 33/0098; G01B 11/02; G01B 11/30; G01N 21/25; H04N 7/183

(57) ABSTRACT

The present invention relates to a method for the determination of the ecological health of a plant by determining the plant seed diversity.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a method of determining the health of a plant seed taxonomic group, i.e. a species or subspecies represented by the plant's seeds. In particular, it is a method of determining Darwinian fitness by determining morphometry of plant seed visual characteristics.

2. Description of Related Art

It is well accepted that Darwinian diversity is the basis on which natural selection of a species operates to bring about evolutionary adaptation for survival, whether the species is wild or domesticated. General observation of this process has concluded that it is likely that an inadequate variation of morphological diversity results in species degradation and survival difficulty, which can and has ultimately led to species extinction. The reduction of morphological diversity that occurs in the domestication of a plant species for commercial use as compared to similar wild type species is well known and mostly due to the intrusion of human selection. Human selection can be arbitrary relative to survival, and is mostly related to commercially desirable traits. This decrease in ecological heath can result in a particular seed taxon being vulnerable to extinction due to a change in disease exposure or climate. In the process of narrowing the variation of plant species in commercial plants, the commercial crops are more susceptible to changes in climatic conditions, diseases, pests and the like. Accordingly, the maintenance, and even the enhancement, of taxonomic diversity contributes to the overall health and survival of an agricultural species.

An agricultural species' seed exhibits morphological variation, as does the grown plant. Variametric analysis of seed populations to determine a relationship between seed morphology and plant survivability has largely been ignored. Conventional analysis of seeds in a species tends to emphasize morphological uniformity and commonality rather than variation. It relies on visual observation, as well as intuitive analysis or a few generally difficult measurements, on relatively small seed populations, leading to bias and widespread systematic measurement errors. Computer-assisted morphometry is a relatively new method, utilizing a digital camera and a computer for assessing the variation in seed populations to determine, for example, whether a seed population is wild or domestic. No further work or speculation was done on this work. Economic Botany, 2007, 61(2), pp 154-172, (hereinafter “Rovner”) incorporated herein by reference in its entirety.

### BRIEF SUMMARY OF THE INVENTION

The present invention relates to the discovery that analysis of the variametric information in a large sample of seeds can determine the relative adaptive health of a species or subspecies (taxon) by determination of the diversity of seed morphology. The greater the diversity, the more healthy the taxon (i.e. the species/subspecies of plant) is.

Accordingly, in one embodiment, there is a method of assessing the ecological health of a specific taxon of a plant comprising:
a) accumulating a large sample of seeds of the specific taxon of the plant;
b) using a digital camera operatively connected to a computer creating visual morphological measurements of the sample of seeds and creating a morphometric database of the measurements on the computer;
c) the computer calculating the seed diversity of the sample from the database: and
d) determining the ecological health of the specific taxon of the plant as being directly related to the diversity of the seeds.

And, in another embodiment, there is a variametric analysis system for determining the ecological health of a specific taxon of a plant from a large sample of seeds comprising:
a) a digital camera operatively connected to a computer capable of creating visual morphological measurements of the sample of seeds and creating a morphometric database of the measurements on the computer;
b) a program in resident memory on the computer for determining the diversity of a large sample of seeds of the plant from the morphological measurements; and
c) a program in resident memory on the computer for calculating the ecological health of the plant from the diversity of the seed sample.

### BRIEF DESCRIPTION OF THE DRAWINGS

FIG. 1 is a diagram of the method of the present invention.

FIG. 2 is a graphic representation of the system of the present invention.

### DETAILED DESCRIPTION OF THE INVENTION

While this invention is susceptible to embodiment in many different forms, there is shown in the drawings, and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitations thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “diversity” refers to the Darwinian diversity that observes the variation within a species exhibits differences.

As used herein the term “morphology” refers to the configuration and/or the condition of observable parameters. In the present invention, the morphology refers to the visual parameters (i.e. morphological visual measurements) visually observable by a digital camera including, but not limited to size, form, color, texture, dimension, tomography, fractals, the associations of characteristics and the like in the biology of a plant (a seed).

As used herein the term “morphometry” refers to the measurement of the morphological visual parameters. Then the morphotype, often from the resulting phenotype, is a construct of selected morphological characteristics, derived from observation by an observer, used to represent members of a taxonomic group, usually a species or subspecies. A metric morphotype is a construct of selected measured morphological characteristics occurring within the range of measured variation, used to represent members of a taxonomic group, usually a species or subspecies.

As used herein the term “variametric analysis” refers to the process and interpretation of the quantitative visual measurement data derived from assemblages of taxonomically related plant seeds.

As used herein the term “ecological health” or “adaptive health” refers generally to the diversity of the seed morphology variation as the plant exists in nature, the field etc., wherein the greater the diversity, the greater the ecological health of the plant. It is then a measurement of the intensity of natural or human selection, the degree of fitness to an associated environment and/or the degree of generalized focus and specialized adaptation. The greater the diversity, then the greater chance for surviving ecological change, thus a greater ecological health. By measuring multiple times over a period of time, the positive or negative direction of ecological health can be measured. While a single morphological parameter can be measured for ecological health, two or more parameters can be utilized.

As used herein the term “single taxon” refers to a specific species or subspecies of a plant represented by the seeds of the plant. In a commercial setting it refers to the species (e.g. a particular variety of commercial tomato) to which general ecological health is important to protect the plant crop from being partially or completely wiped out.

As used herein the term “large sample of seeds” refers to having enough representative seeds in the sample to be able to get a statistically significant result upon measurement of the morphology of the sample. So, in most cases, there needs to be a number of seeds of at least about 50, 100, 200, 300 or the like, in the sample to have a significant amount of seeds.

As used herein the term “digital camera” refers to a camera of the type utilized in the above referenced Rovner article for connecting to a computer and transferring visual data created by the camera to digital information on the computer.

As used herein the term “computer” refers to any type of computing device having a resident memory and software, and includes online, offline, attached and detached memory, and can be a desktop, laptop, or any other type of computer.

As used herein the term “morphometric database of measurements” refers to accumulating the morphometric measurements made with the digital camera in a computer database. The information can be in a standard database, but in one embodiment, the database is a histogram of the morphological data as described above. Using the database of measurements, the computer can then quickly compare each of the seeds morphological data and calculate the seed diversity.

As used herein, the term “determining the ecological health” refers to the process of determining the morphological diversity and the likelihood of health issues based on the degree of seed diversity. So, the more diversity means greater health, the less diversity means less health. It has been discovered that the entire plant's ecological health is determined from just the seed morphology, avoiding the need for time-consuming, difficult, and expensive morphological measurements of the entire plant. It also makes programming morphological differences into a computer easier, since there are less measurements to be made and observed in the seed than in the entire plant.

As used herein the term “commercial plant” refers to a crop being used in industry. This could be an agricultural crop, a cover crop, a forest crop or the like. A wild type plant, on the other hand, is as it would seem—a plant species that is not used in commercial settings and is found wild in nature.

As used herein the term “histogram” refers to general histograms as described above using the morphological data of the present invention. Such histograms are well known in the art, see for example Wikipedia on the term “histogram” incorporated herein by reference.

Darwinian diversity is the basis on which the theory of natural selection operates to bring about evolutionary adaptation in the process of the survival of species. This is true whether the species is wild or domesticated. The result of inadequate variation likely results in the degradation, and even the ultimate extinction, of a species. It is well known that a reduction in the diversity of a domesticated plant compared to their wild relative results from the intrusion of arbitrary and intensified human selection. When there is less variation in a domesticated crop plant it is often detrimental to the health, and thus the viability, of the crop. This lack of variation can come from disease, especially host-specific disease, crop pests and environmental changes. Therefore, the maintenance and enhancement of intrataxon diversity is a positive factor in the “health” of a crop in a sustainable agriculture setting. The measurement of all aspects of a plant's morphology is time-consuming and very difficult. It has been discovered that the morphological diversity of the seeds of a crop plant are sufficient by themselves for assessing the current health of a crop. The morphological diversity of the crop plant seeds can also be used to measure changes in the health of a crop over time and predict vulnerability of a crop to health related factors.

Variametric analysis, using only seed morphometric measurements, provides an objective and quantitative method for evaluating, assessing and monitoring diversity over time, both in wild and domestic crops. This seed specific analysis aids in maintaining the sustainability of and management of domestic crops and wild ecological systems without having to assess the whole plant.

The present invention has studied wild plant seed populations and, using morphometric data of the seeds, shows that patterns of variation are not normally distributed in an overwhelming number of cases tested. They do show diverse, often multimodal, and unpredictable distributions. Domestic crop seed populates are distinct and more predictable (usually under strong human selection), showing comparatively narrow ranges of variation, as well as higher kurtosis values approaching, or even exceeding, normal bell-shaped distributions.

In the present invention, variametric analysis makes no assumptions of Normality or any other standard or expected configuration of variation. Rather, real and objective measurement data are plotted without assumption, for example in one embodiment, in a standard histogram as the basis for analysis. Histograms show the configuration of the distribution of measured variation for a given morphological parameter. Rather than assessing the probabilities, as in statistical analysis, a histogram is treated as an irregular object of varying unpredictable shape. The histogram is then treated as a digital or irregular object and exported to a morphometric computer program where it is objectively quantified, i.e. measured, using standard calculated shape factor algorithms. Among the standard shape factors used to measure histograms, three thus far appear useful in evaluating size measurement histograms: Form Factor, Convexity and Curl. These consistently yield lower values for multimodal (i.e. wild plant) seed histograms and higher values for normal, near normal and hypernormal (i.e. domestic plant) seed histograms. The resulting shape measurements are then used as a basis for systematic identifications and comparisons of seeds, including confirmation and verification of taxonomic classifications. Variametric analysis also presents quantitative data reflecting conditions of Darwinian factors as an extent of morphological variation, intensity of natural selection, degree of fitness to an associated environment and degree of generalized versus specialized adaptation.

As an example, Darwinian Variation as generated by genetic mutation, Mendelian Free Recombination, and the like, and natural or human selection are known to be opposing forces in nature. Selection tends to eliminate “less fit” variants of a population, leaving space for an increase in variants more fit to current conditions, thus narrowing overall diversity. In a simulated idealized nature, where only selection operates (i.e. 100% selection and 0% mutation), complete elimination of less fit variants would result in a uniform population of only the single most fit variant. The histogram of measurement of such a population would be a single vertical line parallel to the y-axis on a standard X Y chart or graph. Alternately, in an idealized nature, where only mutation operates (i.e. 0% selection and 100% mutation) every viable variant is equally fit and successful compared to all other variants. Ideally, the frequency of each variant would be random and equal to every other variant. The resulting histogram would show a horizontal line parallel to the X-axis. In the real world, however, the histogram showing the measurement of a morphological parameter is a quantitative bivariate plot showing the relative effects of Darwinian selection (amplitudes of modes on the Y-axis) versus the distribution of variation (distribution on the X-axis). Subjecting the histogram to morphometric analysis provides objective, quantitative measure of Darwinian selection adaptability, fitness and diversity or variation.

Variametric analysis then provides objective, and quantitative comparisons of diversity, providing a measure of critical conditions i.e. the health of an agricultural crop species or variety, with respect to natural crop defenses or the general health of crops in a wild condition (such as a reserve). Variametric analysis can be utilized to measure the intensity of human selection in commercial crops and the vulnerability to failure in the face of crop pressure, such as disease, pests or climate variation. Variametric analysis of morphometric data of seeds is then a direct proxy for changes in genetic diversity in the crop itself, providing a cost-effective method for assessment of the quality and quantity of genetic diversity, allowing focused and efficient application of measurements. Results from variametric analysis can then be used to monitor efforts to improve intraspecies crop diversity, and can be applied to the implementation of ecologically friendly procedures in support of improved natural sustainability. Accordingly, the present process will have global application for ecological, agricultural and economic factors in the management of crop resources.

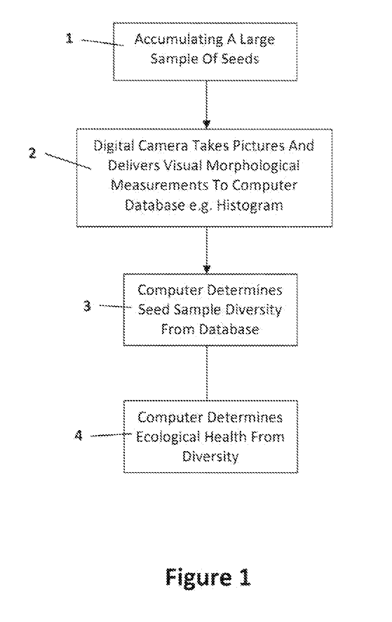

Now referring to the drawings, FIG. 1 is a flow chart of the method of the present invention. As shown, the method starts with an accumulation of a large sample of seeds 1 of a particular commercial or wild type plant. Next, a digital picture of each seed is made 2, and this visual information of the morphological measurements is sent to a computer database in any form, such as a histogram.

The computer takes the measured seed morphological data and determines the seed sample diversity 3 (determining if there is a large diversity or a narrow diversity etc.). Finally, the computer determines the ecological health from the diversity 4. It can do that from a single sample or make multiple readings over time to get not only the ecological health, but also the direction the health is moving, as described above.

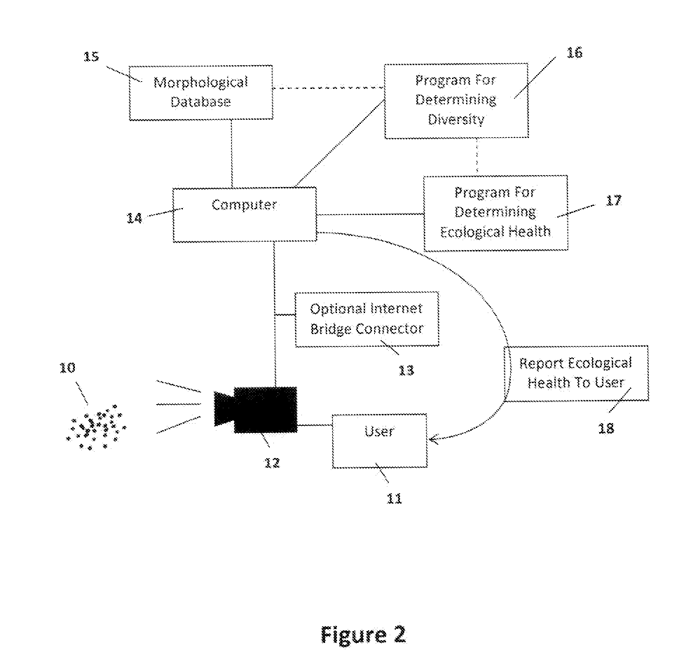

FIG. 2 is a chart showing the relationship of the parts of the system of the present invention. A large sample of seeds (about 50 or higher) 10 is photographed by a digital camera 12 for measurement of morphological visual characteristics by a user 11. The visual information on digital camera 12 is sent to a computer 14 and, in one embodiment, via an internet connection 13. From there, the information is kept in a morphological measurement database 15, and a program on the computer determines diversity 16 by comparing each of the measurements for each seed measured. The same or different program then determines the ecological health 17 of the plant by reviewing the diversity of the seeds. In one embodiment, this process is repeated one or more times to get a direction to the health. In each case, the results of the determination of health 17 are reported to the user 18. The user can then use that data to decide if anything should be done regarding the health of the plant, for example, better pest and disease treatment, selection of a different species to grow commercially, greater monitoring of the crops or wild environments, optimum germination zone, seed size, and selection for efficient water usage and the like.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art, these still fall within the scope of the invention as claimed by the Applicant.

What is claimed is:

1. A method of assessing the ecological health of a specific taxon of a plant comprising:
a) accumulating a large sample of seeds of the specific taxon of the plant;
b) using a digital camera operatively connected to a computer creating visual morphological measurements of the sample of seeds and creating a morphometric database of the measurements on the computer;
c) the computer calculating the seed diversity of the sample from the database: and
d) Determining the ecological health of the specific taxon of the plant as being directly related to the diversity of the seeds.

2. The method according to claim 1 wherein the sample of seeds is at least about 100 seeds.

3. The method according to claim 1 wherein the morphometric measurements consists of at least one of size, dimension, color, tomography, texture, and fractals.

4. The method according to claim 1 wherein the method is repeated over time, each time with a new sample of seeds and the change in the health of the specific taxon of a plant is determined.

5. The method according to claim 1 wherein the ecological health of the specific taxon of a commercial plant is used to determine if the specific taxon should be changed in a commercial setting.

6. The method according to claim 1 wherein the health of the specific taxon of the commercial plant is used to determine procedures for growing the commercial plant for improved sustainability.

7. The method according to claim 1 wherein the plant is a commercial plant.

8. The method according to claim 1 wherein the plant is a wild type plant.

9. The method according to claim 1 wherein a commercial plant's health is compared to the health of a wild type plant of the same species.

10. The method according to claim 1 wherein the database is populated with a histogram of the data.

11. The method according to claim 1 wherein the method measures at least one of the group consisting of morphological variation, intensity of natural selection, degree of fitness to an associated environment and generalized versus specialized adaptation.

12. A variametric analysis system for determining the ecological health of a specific taxon of a plant from a large sample of seeds comprising:
a) a digital camera operatively connected to a computer capable of creating visual morphological measurements of the sample of seeds and creating a morphometric database of the measurements on the computer;
b) a program in resident memory on the computer for determining the diversity of a large sample of seeds of the plant from the morphological measurements; and
c) a program in resident memory on the computer for calculating the ecological health of the plant from the diversity of the seed sample.

13. An analysis system according to claim 12 which further comprises a database of wild type seed diversity for comparison to the health of a commercial crop of the same species.
